(19) United States
(12) Patent Application Publication
(10) Pub. No.: US 2024/0033041 A1
(43) Pub. Date: Feb. 1, 2024

(54) METHOD OF DETERMINING TOOTH ROOT APICES USING INTRAORAL SCANS AND PANORAMIC RADIOGRAPHS

(71) Applicant: Align Technology, Inc., San Jose, CA (US)

(72) Inventors: Vitaliy Vladimirovich CHERNOV, Madrid (ES); Egor Artemovich KHROMOV, Yerevan (AM); Mikhail Nikolaevich RYCHAGOV, Darmstadt (DE)

(21) Appl. No.: 18/358,913

(22) Filed: Jul. 25, 2023

Related U.S. Application Data
(60) Provisional application No. 63/392,447, filed on Jul. 26, 2022.

(51) Int. Cl. A61C 7/00; G06T 7/00

(52) U.S. Cl. CPC A61C 7/002; G06T 7/00; G06T 2207/30036; G06T 2207/20081; G06T 2207/20084

(57) ABSTRACT

Methods, apparatuses, and systems are disclosed for determining accurate tooth root apices from two-dimensional panoramic radiograph data and/or three-dimensional intraoral scan data. In some variations, one or more deep learning networks may be trained to determine coordinates of tooth root apices based on training data that may include cone beam computer tomography tooth data and corresponding two-dimensional panoramic radiograph data and three-dimensional intraoral scan data.

### CLAIM OF PRIORITY

This patent application claims priority to U.S. Provisional Patent Application No. 63/392,447, titled “METHOD OF DETERMINING TOOTH ROOT APICES USING INTRAORAL SCANS AND PANORAMIC RADIOGRAPHS,” filed on Jul. 26, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The systems and methods described herein relate generally to the generation of dental appliances, and more particularly to predicting the tooth root apices of a patient's teeth to improve the outcome and predictability of an orthodontic and/or dental treatment through the use of a dental appliance.

### BACKGROUND

Treatment planning may be used in any medical procedure to help guide a desired outcome. For example, treatment planning may be used in orthodontic and dental treatments using a series of patient-removable appliances (e.g., orthodontic aligners, palatal expanders, etc.) are very useful for treating patients, and in particular for treating malocclusions. Treatment planning is typically performed in conjunction with the dental professional (e.g., dentist, orthodontist, dental technician, etc.), by generating a model of the patient's teeth in a final configuration and then breaking the treatment plan into a number of intermediate stages (steps) corresponding to individual appliances that are worn sequentially. This process may be interactive, adjusting the staging and in some cases the final target position, based on constraints on the movement of the teeth and the dental professional's preferences.

Successful treatment planning may benefit from accurate estimation of the apices of the roots one or more of the patient's teeth. However, conventional non-invasive procedures to determine root apices may not provide adequate accuracy, may be computationally intensive and are not sufficiently accurate.

### SUMMARY OF THE DISCLOSURE

Described herein are apparatuses, systems, and methods to determine coordinates of apices of a patient's tooth roots. The apices may be determined with limited patient information that may include two-dimensional (2D) panoramic radiograph data or three-dimensional (3D) intraoral scan data. Also described are deep learning convolutional neural networks that may be trained to determine coordinates of the tooth root apices.

Any of the methods described herein may be used to determine coordinates of one or more tooth root apex. Any of the methods may include obtaining patient data, wherein the patient data includes at least one of 2D panoramic radiograph data of a patient and 3D intraoral scan data of the patient, obtaining a tooth number, providing the patient data and the tooth number to a deep learning network, and determining, via a processor executing the deep learning network, the coordinates of a tooth root apex based on the patient data and corresponding to the tooth number.

In any of the methods described herein, the deep learning network may include a 2D convolutional neural network configured to determine the coordinates of the tooth root apex from the 2D panoramic radiograph data of the patient. In some methods, the 2D convolutional neural network may be trained based at least in part on cone beam computed tomography (CBCT) tooth data.

In any of the methods described herein, the deep learning network may include a 3D convolutional neural network configured to determine the coordinates of the tooth root apex from the 3D intraoral scan data of the patient. In some methods, the 3D convolutional neural network may be trained based at least in part on CBCT tooth data.

In any of the methods described herein, the tooth number may selectively weight an output of the deep learning network. Furthermore, in any of the methods the deep learning network may determine the coordinates of the tooth root apex based on the 2D panoramic radiograph data of the patient and the 3D intraoral scan data of the patient.

In any of the methods described herein, the deep learning network may be trained based at least in part on cone beam computed tomography (CBCT) tooth data and 2D panoramic radiograph data corresponding to the CBCT tooth data. In any of the methods described herein, the deep learning network may be trained based at least in part on CBCT tooth data and 3D intraoral scan data corresponding to the CBCT tooth data.

Any of the systems described herein may be used to determine one or more coordinates of a tooth root apex. The system may include a treatment plan generator engine and a processor. The treatment plan generator may be configured to obtain, from a memory, patient data, wherein the patient data includes at least one of two-dimensional (2D) panoramic radiograph data of a patient and three-dimensional (3D) intraoral scan data of the patient, obtain, from the memory, a tooth number, and provide the patient data and the tooth number to a deep learning network. The processor may be configured to determine, via the deep learning network, coordinates of a tooth root apex based on the patient data and corresponding to the tooth number.

Any of the non-transitory computer-readable storage mediums described herein may include instructions that, when executed by one or more processors of a device, cause the device to perform operations comprising obtaining patient data, wherein the patient data includes at least one of two-dimensional (2D) panoramic radiograph data of a patient and three-dimensional (3D) intraoral scan data of the patient, obtaining a tooth number, providing the patient data and the tooth number to a deep learning network, and determining, via a processor executing the deep learning network, the coordinates of a tooth root apex based on the patient data and corresponding to the tooth number.

Any of the methods described herein may be used to train a deep learning network to determine coordinates of tooth root apices. The method may include obtaining cone beam computed tomography (CBCT) tooth data, obtaining two-dimensional (2D) panoramic radiograph data corresponding to the CBCT tooth data, obtaining three-dimensional (3D) intraoral scan data corresponding to the CBCT tooth data, and training, via a treatment planning engine, a deep learning network to determine coordinates of tooth root apices of a patient.

In any of the methods described herein, the CBCT tooth data may be used as ground truth data during the training. Furthermore, the training may include minimizing a cost function associated with determined tooth root apices of the patient and the ground truth data.

In any of the methods described herein, the CBCT tooth data may include segmented 3D voxel data. Furthermore, the segmented 3D voxel data may include tooth root apex coordinates.

In any of the methods described herein, the CBCT tooth data may include tooth number identification data. In any of the methods described herein, the training may include training a 2D convolutional neural network to determine the coordinates of the tooth root apices of the patient based on 2D panoramic radiograph data of the patient. Furthermore, training data for the 2D convolutional neural network may include the CBCT tooth data, the 2D panoramic radiograph data, and the 3D intraoral scan data.

In any of the methods described herein, the training may include training a 3D convolutional neural network to determine the coordinates of the tooth root apices of the patient based on 3D intraoral scan data of the patient. Furthermore, training data for the 3D convolutional neural network may include the CBCT tooth data, the 2D panoramic radiograph data, and the 3D intraoral scan data.

In any of the methods described herein, tooth apex coordinates of the CBCT tooth data, the 2D panoramic radiograph data, and the 3D intraoral scan data may be each within a predetermined distance of each other.

Any of the systems described herein may be used for determining coordinates of a tooth root apex. The system may include a treatment plan generator engine configured to obtain cone beam computed tomography (CBCT) tooth data, obtain two-dimensional (2D) panoramic radiograph data corresponding to the CBCT tooth data, obtain three-dimensional (3D) intraoral scan data corresponding to the CBCT tooth data; and training, via a treatment planning engine, a deep learning network to determine coordinates of tooth root apices of a patient.

Any of the non-transitory computer-readable storage mediums described herein may include instructions that, when executed by one or more processors of a device, cause the device to perform operations comprising obtaining cone beam computed tomography (CBCT) tooth data, obtaining two-dimensional (2D) panoramic radiograph data corresponding to the CBCT tooth data, obtaining three-dimensional (3D) intraoral scan data corresponding to the CBCT tooth data, and training, via a treatment planning engine, a deep learning network to determine coordinates of tooth root apices of a patient.

For example, described herein are methods of determining coordinates of a tooth root apex. A method of determining coordinates of a tooth root apex may include: obtaining patient data, wherein the patient data includes at least one of two-dimensional (2D) panoramic radiograph data of a patient and three-dimensional (3D) intraoral scan data of the patient; obtaining a tooth number; providing the patient data and the tooth number to a deep learning network; and determining, via a processor executing the deep learning network, the coordinates of a tooth root apex based on the patient data and corresponding to the tooth number.

The deep learning network may include a 2D convolutional neural network configured to determine the coordinates of the tooth root apex from the 2D panoramic radiograph data of the patient. The 2D convolutional neural network may be trained based at least in part on cone beam computed tomography (CBCT) tooth data. In some examples the deep learning network includes a 3D convolutional neural network configured to determine the coordinates of the tooth root apex from the 3D intraoral scan data of the patient.

The 3D convolutional neural network may be trained based at least in part on CBCT tooth data.

The tooth number may selectively weight an output of the deep learning network.

In some examples the deep learning network determines the coordinates of the tooth root apex based on the 2D panoramic radiograph data of the patient and the 3D intraoral scan data of the patient.

The deep learning network may be trained based at least in part on cone beam computed tomography (CBCT) tooth data and 2D panoramic radiograph data corresponding to the CBCT tooth data. For example, the deep learning network may be trained based at least in part on CBCT tooth data and 3D intraoral scan data corresponding to the CBCT tooth data.

Also described herein are systems for performing any of the methods described herein. For example a system for determining coordinates of a tooth root apex may include: a treatment plan generator engine configured to: obtain, from a memory, patient data, wherein the patient data includes at least one of two-dimensional (2D) panoramic radiograph data of a patient and three-dimensional (3D) intraoral scan data of the patient; obtain, from the memory, a tooth number; provide the patient data and the tooth number to a deep learning network; and a processor configured to determine, via the deep learning network, coordinates of a tooth root apex based on the patient data and corresponding to the tooth number.

Also described herein is software for performing any of these methods (e.g., non-transitory computer-readable storage medium comprising instructions for performing any of these methods). For example, described herein are non-transitory computer-readable storage medium comprising instructions that, when executed by one or more processors of a device, cause the device to perform operations comprising: obtaining patient data, wherein the patient data includes at least one of two-dimensional (2D) panoramic radiograph data of a patient and three-dimensional (3D) intraoral scan data of the patient; obtaining a tooth number; providing the patient data and the tooth number to a deep learning network; and determining, via a processor executing the deep learning network, coordinates of a tooth root apex based on the patient data and corresponding to the tooth number.

Also described herein are methods of training a deep learning network to determine coordinates of tooth root apices, the method comprising: obtaining cone beam computed tomography (CBCT) tooth data; obtaining two-dimensional (2D) panoramic radiograph data corresponding to the CBCT tooth data; obtaining three-dimensional (3D) intraoral scan data corresponding to the CBCT tooth data; and training, via a treatment planning engine, a deep learning network to determine coordinates of tooth root apices of a patient.

The CBCT tooth data may be used as ground truth data during the training. The training may include minimizing a cost function associated with determined tooth root apices of the patient and the ground truth data. In some examples the CBCT tooth data includes segmented 3D voxel data. The segmented 3D voxel data may include tooth root apex coordinates. In some examples the CBCT tooth data includes tooth number identification data. The training may include training a 2D convolutional neural network to determine the coordinates of the tooth root apices of the patient based on 2D panoramic radiograph data of the patient.

In some examples training data for the 2D convolutional neural network includes the CBCT tooth data, the 2D panoramic radiograph data, and the 3D intraoral scan data. For example, the training may include training a 3D convolutional neural network to determine the coordinates of the tooth root apices of the patient based on 3D intraoral scan data of the patient. In some examples training data for the 3D convolutional neural network includes the CBCT tooth data, the 2D panoramic radiograph data, and the 3D intraoral scan data.

The tooth apex coordinates of the CBCT tooth data, the 2D panoramic radiograph data, and the 3D intraoral scan data may each be within a predetermined distance of each other.

Also described herein are system for determining coordinates of a tooth root apex, the system comprising: a treatment plan generator engine configured to: obtain cone beam computed tomography (CBCT) tooth data; obtain two-dimensional (2D) panoramic radiograph data corresponding to the CBCT tooth data; obtain three-dimensional (3D) intraoral scan data corresponding to the CBCT tooth data; and training, via a treatment planning engine, a deep learning network to determine coordinates of tooth root apices of a patient.

Also described herein are non-transitory computer-readable storage medium comprising instructions that, when executed by one or more processors of a device, cause the device to perform operations comprising: obtaining cone beam computed tomography (CBCT) tooth data; obtaining two-dimensional (2D) panoramic radiograph data corresponding to the CBCT tooth data; obtaining three-dimensional (3D) intraoral scan data corresponding to the CBCT tooth data; and training, via a treatment planning engine, a deep learning network to determine coordinates of tooth root apices of a patient.

In general, the methods and apparatuses (e.g., systems) described herein may be configured to formulate and/or modify a treatment plan (e.g., an orthodontic treatment plan) and/or may be used to fabricate one or more dental appliance suing these treatment plans.

For example, described herein are methods of generating a treatment plan for forming one or more dental appliances by determining coordinates of a tooth root apex. These methods may include: obtaining patient data, wherein the patient data includes at least one of two-dimensional (2D) panoramic radiograph data of a patient and three-dimensional (3D) intraoral scan data of the patient; obtaining a tooth number; providing the patient data and the tooth number to a trained deep learning network; and determining, via a processor executing the trained deep learning network, the coordinates of a tooth root apex based on the patient data and corresponding to the tooth number; generating or modifying the treatment plan using the coordinates of the tooth root apex; and forming one or more dental appliances according to the treatment plan.

As used herein a dental appliance may be formed by forming the dental appliance digitally (e.g., by generating digital plans, including but not limited to schematics) in sufficient detail so that a physical dental appliance may be formed, including (but not limited to) 3D printing or other similar techniques. In some examples, the dental treatment plan may include sufficient detail to fabricate the dental appliance. In some examples the method or apparatuses described herein may generate a digital file that may be read by, received and our operated upon by a 3D printer.

As mentioned above, any of these methods and apparatuses may be configured as a system, e.g., a system for determining coordinates of a tooth root apex. For example such a system may include: a treatment plan generator engine configured to: obtain, from a memory, patient data, wherein the patient data includes at least one of two-dimensional (2D) panoramic radiograph data of a patient and three-dimensional (3D) intraoral scan data of the patient; obtain, from the memory, a tooth number; and provide the patient data and the tooth number to a trained deep learning network; and a processor configured to determine, via the trained deep learning network, coordinates of a tooth root apex based on the patient data and corresponding to the tooth number, wherein the treatment plan generator is configured to use the coordinates of the tooth root apex to generate or modify a treatment plan.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a diagram showing an example of systems in a device planning environment.

FIG. 2 is a diagram showing an example of a system.

FIG. 3 is a flowchart showing an example method for training a deep learning network for determining coordinates of tooth root apices.

FIG. 4 shows an example of an intraoral scan matched with a segmented CBCT scan.

FIG. 5 is a block diagram showing data and process flow of a deep learning network configured to determine tooth root apex coordinates of a patient's teeth.

FIG. 6. is a flowchart showing an example method for determining tooth root apex coordinates via a deep learning network.

FIG. 7 is a block diagram showing data and process flow of a deep learning network configured to determine tooth root apex coordinates of a patient's teeth.

FIG. 8. is a flowchart showing an example method for determining tooth root apex coordinates via a deep learning network.

FIG. 9 shows a block diagram of device that may be one example of a device that may be configured to perform any of the operations described herein.

### DETAILED DESCRIPTION

Dental treatment planning may be easier and more effective when information regarding the root structure of the patient's teeth are available. To get information about the roots of the patient's teeth, clinicians may take panoramic radiographs (pantomograms), lateral cephalograms, full-mouth x-rays as well as cone beam computed tomography (CBCT) images. CBCT images may provide the greatest amount of information regarding invisible parts of the patient's teeth due to its three-dimensional nature. However, in most of circumstances, CBCT images of the patient are not available. CBCT equipment may be expensive and therefore difficult for a clinician to access. In some cases, the patient may be reticent to receive the x-ray exposure associated with CBCT scans.

In some examples, accurate tooth root information, including tooth root apex information, may be determined using patient data that includes two-dimensional panoramic radiograph data and/or three-dimensional intraoral scan data or a patient's teeth. The patient data may be processed using a machine learning based deep learning network. The deep learning network may be trained using CBCT data as well as corresponding two-dimensional (2D) panoramic radiograph data and three-dimensional (3D) intraoral scan data.

After the deep learning network has been trained, a clinician may provide a patient's 2D panoramic radiograph data and/or 3D intraoral scan data to the deep learning network. In some variations, the clinician may also provide a tooth number of a particular tooth. The deep learning network may provide tooth root apex information based on the 2D panoramic radiograph data, the 3D intraoral scan data, and tooth number. The tooth root apex information may include coordinate information of apices of a plurality of tooth roots. The tooth root apex information may be used to determine dental treatment plans for the patient.

The deep learning network may advantageously provide tooth root apex information without the need for CBCT scan information or CBCT equipment.

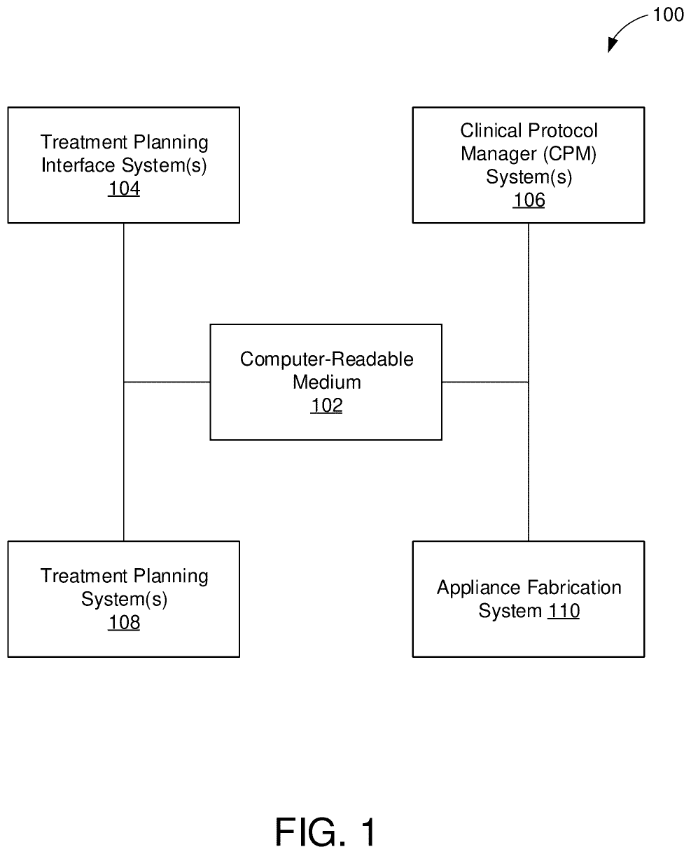

FIG. 1 is a diagram showing an example of systems in a device planning environment 100. The device planning environment 100 may include a computer-readable medium 102, treatment planning interface system(s) 104, a clinical protocol manager (CPM) system(s) 106, treatment planning system(s) 108, and appliance fabrication system(s) 110. One or more of the components (including modules) of the device planning environment 100 may be coupled to one another (e.g., through the example couplings shown in FIG. 1) or to modules not explicitly shown in FIG. 1. The computer-readable medium 102 may include any computer-readable medium, including without limitation a bus, a wired network, a wireless network, or some combination thereof.

A computer system can be implemented as an engine, as part of an engine or through multiple engines. As used herein, an engine may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices and need not be restricted to only one computing device. In some embodiments, the cloud-based engines can execute functionalities and/or modules that end users access through a web browser or container application without having the functionalities and/or modules installed locally on the end-users' computing devices.

As used herein, datastores (e.g., data bases or other warehouses of data) are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The treatment planning interface system(s) 104 may include one or more computer systems configured to interact with users and provide users with the ability to manage treatment plans for patients. A “user,” in this context, may refer to any individual who can access and/or use the treatment planning interface system(s) 104, and can include any medical professional, including dentists, orthodontists, podiatrists, medical doctors, surgeons, clinicians, etc.

In some implementations, the treatment planning interface system(s) 104 includes engines to gather patient data related to patients who are to be treated according to a treatment plan.

“Patient data,” as used herein, may include data related to a patient. Patient data may include representations of anatomical information, such as information about specific portions of the human body to be treated. Examples of anatomical information include representations of a patient's dentition, bones, organs, etc. at a specific time. Patient data may represent anatomical information before, during, or after a treatment plan. As examples, patient data may represent the state and/or intended state of a patient's dentition before, during, or after orthodontic or restorative treatment plans. Patient data may be captured using a variety of techniques, including from a scan, digitized impression, etc. of the patient's anatomy. Additionally, or alternatively, patient data may include patient oral scan information. For example, patient information may include 2D panoramic radiograph data and/or 3D intraoral scan data of a patient's teeth and (in some cases) surrounding bone structure.

A “treatment plan,” as used herein, may include a set of instructions to treat a medical condition. A treatment plan may specify, without limitation treatment goals, specific appliances used to implement the goals, milestones to measure progress, and other information, such as treatment length and/or treatment costs. As noted herein, in some implementations, the treatment planning interface system(s) 104 provides a user with an orthodontic treatment plan to treat malocclusions of teeth. The treatment planning interface system(s) 104 may also provide users with restorative treatment plans for a patient's dentition and other types of medical treatment plans to address medical conditions patients may have. In some implementations, a treatment plan may include an automated and/or real-time treatment plan, such as the treatment plans described in U.S. patent application Ser. No. 16/178,491, entitled “Automated Treatment Planning,” the contents of which are incorporated by reference as if set forth fully herein. A treatment plan may also include treatment instructions provided by a treatment technician, such as a treatment technician who provides the treatment plan to the user of the treatment planning interface system(s) 104 through the computer-readable medium 102.

In various implementations, the treatment planning interface system(s) 104 is configured to allow a user to visualize, interact with, and/or fabricate appliances that implement a treatment plan. As an example, the treatment planning interface system(s) 104 may provide a user with a user interface that displays virtual representations of orthodontic appliances that move a patient's teeth from an initial position toward a final position to correct malocclusions of teeth. The treatment planning interface system(s) 104 can similarly display representations of restorative appliances and/or other medical appliances. The treatment planning interface system(s) 104 may allow a user to modify appliances through a UI supported thereon. In various implementations, the treatment planning interface system(s) 104 allows a user to fabricate appliances through, e.g., the appliance fabrication system(s) 110. (It is noted the appliance fabrication system(s) 110 may but need not be remote to the treatment planning interface system(s) 104 and can be located proximate to the treatment planning interface system(s) 104.)

The treatment planning interface system(s) 104 may be configured to provide a user with UIs that allow the user to discuss treatment plans with patients. As an example, the treatment planning interface system(s) 104 may display to the user portions of patient data (e.g., depictions of a condition to be treated) as well as treatment options to correct a condition. The treatment planning interface system(s) 104 may display potential appliances that are prescribed to implement the treatment plan. As an example, the treatment planning interface system(s) 104 may display to the user a series of orthodontic appliances that are configured to move a patient's dentition from a first position toward a target position in accordance with an orthodontic treatment plan. The treatment planning interface system(s) 104 may further be configured to depict the effects of specific appliances at various stages of a treatment plan.

The treatment planning interface system(s) 104 may be configured to allow a user to interact with a treatment plan. In some implementations, the treatment planning interface system(s) 104 allows a user to specify treatment preferences. “Treatment preferences,” as used herein, may include specific treatment options and/or treatment tools that a user prefers when treating a condition. Treatment preferences may include clinical settings, treatment goals, appliance attributes, preferred ranges of movement, specific stages to implement a specific procedure, etc. Examples of clinical settings in an orthodontic context include allowing or disallowing a type of treatment, use of various types of movements on specific teeth (e.g., molars), use of specific procedures (e.g., interproximal reduction (IPR)), use of orthodontic attachments on specific teeth, etc. Examples of treatment goals in an orthodontic context include lengths/costs of treatments, specific intended final and/or intermediate positions of teeth, etc. Example ranges of movement in an orthodontic context include specific distances and/or angles teeth are to move over various stages of treatment and/or specific forces to be put on teeth over various stages of treatment. Specific stages to implement a specific procedure include, for instance in the orthodontic context, a specific treatment stage to implement attachments, hooks, bite ramps and/or to perform procedures such as surgery or interproximal reduction.

As discussed further herein, the treatment planning interface system(s) 104 may be configured to provide users with customized GUI elements based on treatment templates that structure their treatment preferences in a manner that is convenient to them. Customized GUI elements may include forms, text boxes, UI buttons, selectable UI elements, etc.). In some implementations, customized GUI elements may list treatment preferences and provide a user with the ability to accept, deny, and/or modify treatment preferences. Customized GUI elements may provide the ability to accept or deny parts of at treatment plan and/or modify portions of a treatment plan. In some implementations, a user's customized GUI elements provide the ability to modify parts of an appliance recommended for a treatment plan. For instance, a treatment-related UI element may provide the ability to modify force systems, velocities of tooth movement, angles and/or orientations of parts of aligners, crowns, veneers, etc. that are implemented at specific stages of an orthodontic or restorative treatment plan.

“Treatment templates,” as used herein, may include structured data expressed in “treatment domain-specific protocols.” (In some examples, treatment templates are generated by the CPM system(s) 106, stored in datastores on the treatment planning system(s) 108, and parsed by engines on the treatment planning system(s) 108 that create customized GUI elements on the treatment planning interface system(s) 104.)

“Treatment domain-specific protocols,” as used herein, may include computer languages, runtime objects (e.g., applications, processes, etc.), interpreted items (e.g., executed scripts), etc. that are specialized to treatment planning. Treatment domain-specific protocols may include attributes that are specialized to patient data and/or the gathering thereof, attributes that are specialized to description and/or interaction with treatment plans, and attributes that are specialized to appliances used to implement a treatment plan. The present disclosure provides a detailed example of orthodontic domain-specific protocols. It is noted the examples herein may apply to restorative and/or dental domain-specific protocols and other medical domain-specific protocols.

In some implementations, treatment templates include customized graphical user interface (GUI) elements. Customized GUI elements may be generated using treatment domain-specific protocols. As noted herein, the treatment templates for a user may be customized based on a template library of treatment templates for other users. As an example, a treatment template for a user may be derived from and/or otherwise based on a treatment template of another user (e.g., the treatment preferences in that treatment template may be derived from and/or otherwise based on treatment preferences of another user). Public templates may provide the basis of deriving treatment preferences of other users. Private templates may provide a basis of deriving treatment preferences of a specific user. Additionally, customized GUI elements may be automatically generated during execution of applications and/or processes on the treatment planning interface system(s) 104. Customized GUI elements may operate to display attributes of treatment plans that are relevant to a specific user.

The CPM system(s) 106 may include one or more computer systems configured to create treatment templates using treatment domain-specific protocols. In some implementations, the CPM system(s) 106 are operated by CPM technicians, who may, but need not, be remote to users of the treatment planning interface system(s) 104. The CPM system(s) 106 may also be operated by automated agents. The CPM system(s) 106 may include tools to create treatment templates for specific users based on unstructured representations of treatment preferences of those users. In some implementations, the CPM system(s) 106 are configured to obtain past treatment preferences for users through telephonic interviews, emails, notes memorializing discussions, etc. The CPM system(s) 106 may provide technicians with editing tools to structure treatment preferences in a manner that can be organized for a treatment domain-specific protocol. In various implementations, the CPM system(s) 106 are configured to support creating and editing of treatment domain-specific protocols. As an example, the CPM system(s) 106 may be configured to allow technicians to create and/or edit treatment domain-specific scripts that structure treatment preferences for a specific user.

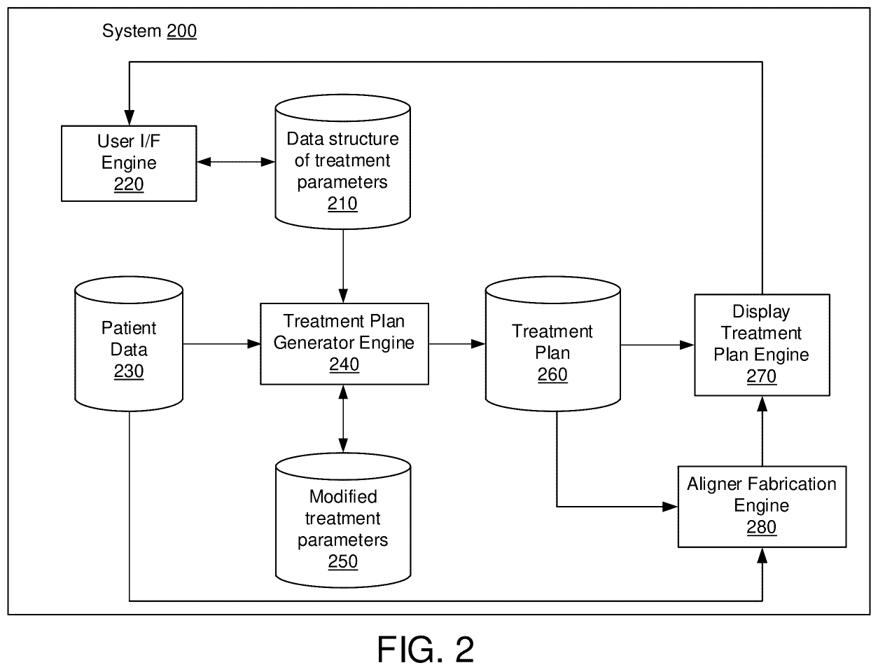

Additionally, the CPM system(s) 106 may provide validation tools to validate treatment domain-specific protocols to ensure the treatment domain-specific protocols are accurate or otherwise in line with treatment preferences. As an example, the CPM system(s) 106 may provide a visual depiction of how specific treatment domain-specific protocols would appear in treatment planning software. As noted herein, the CPM system(s) 106 may employ one or more validation metrics to quantify validation. Examples of validation metrics that may be relevant to an orthodontic context include arch expansion metrics per quadrant, overjet metrics, overbite metrics, interincisal angle metrics, and/or flags if a treatment plan conforms with minimal or threshold root movement protocols. The CPM system(s) 106 may include one or more elements of the system 200 shown in FIG. 2.

The treatment planning system(s) 108 may include one or more computer systems configured to provide treatment plans to the treatment planning interface system(s) 104. The treatment planning system(s) 108 may receive patient data and the treatment preferences relevant to a user. The treatment planning system(s) 108 may further provide treatment plans for the patient data that accommodate the treatment preferences relevant to the user. The treatment planning system(s) 108 may implement automated and/or real-time treatment planning as referenced further herein.

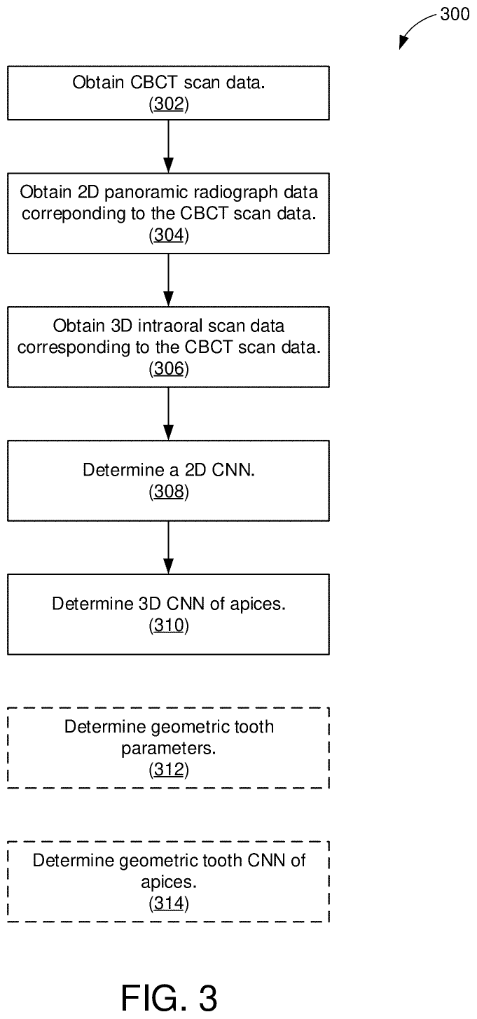

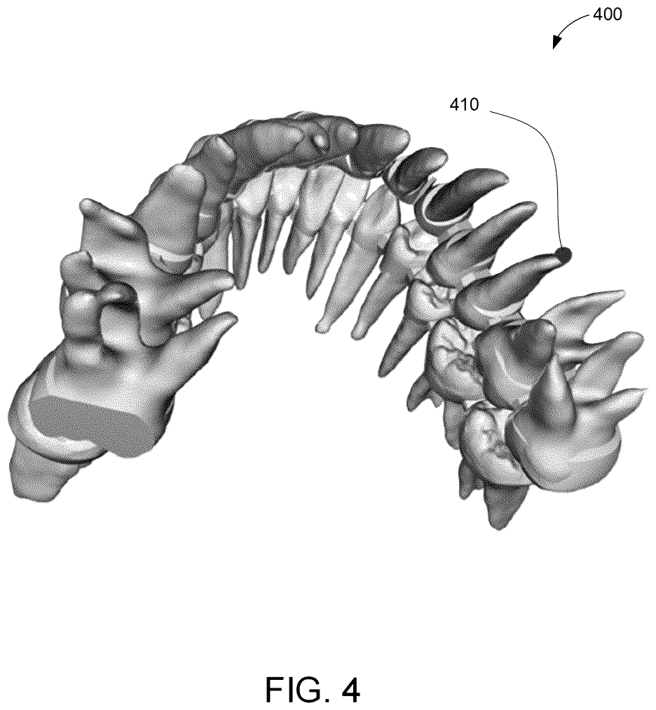

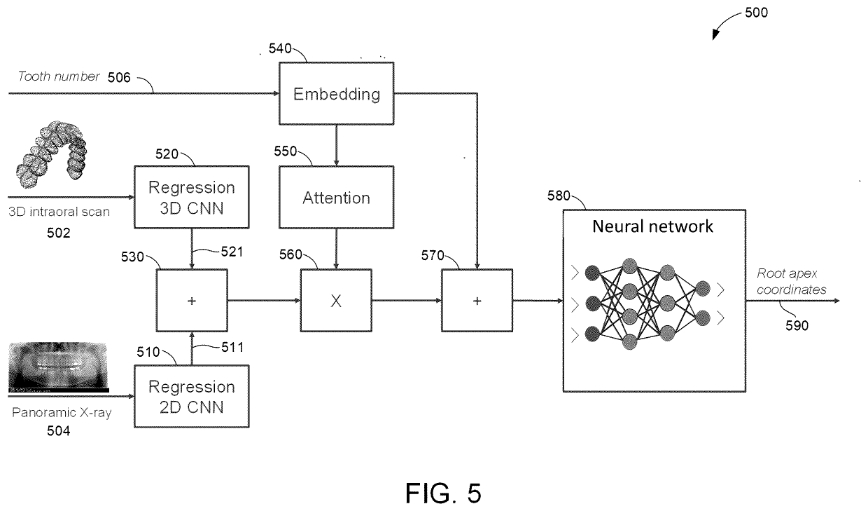

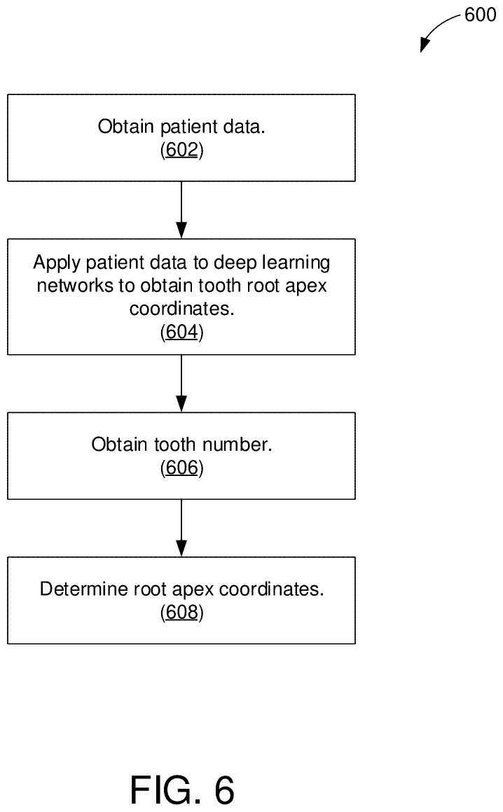

The treatment planning system(s) 108 may include one or more engines configured train one or more deep learning networks. The deep learning networks may be trained to determine tooth root characteristics, including apex information associated with a patient's teeth. Training deep learning networks is described in more detail in conjunction with FIGS. 3 and 4. In some examples, the treatment planning system(s) 108 may also include one or more engines configured to execute the one or more deep learning networks. Thus, the treatment planning system(s) 108 may determine tooth root characteristics regarding a patient's teeth. Execution of deep learning networks is described in more detail below in conjunction with FIGS. 5 and 6.

The treatment planning system(s) 108 may include one or more engines configured to provide treatment plans to the treatment planning interface system(s) 104. The treatment planning system(s) 108 may receive patient data and the treatment preferences relevant to a user. The treatment planning system(s) 108 may further provide treatment plans for the patient data that accommodate the treatment preferences relevant to the user. In various implementations, the treatment planning system(s) 108 identify and/or calculate treatment plans with instructions treat medical conditions. The treatment plans may specify treatment goals, specific outcomes, intermediate outcomes, and/or recommended appliances used to achieve goals/outcomes. The treatment plan may also include treatment lengths and/or milestones. In various implementations, the treatment planning system(s) 108 calculate orthodontic treatment plans to treat malocclusions of teeth, restorative treatment plans for a patient's dentition, medical treatment plans, etc. The treatment plan may comprise automated and/or real-time elements and may include techniques described in U.S. patent application Ser. No. 16/178,491, entitled “Automated Treatment Planning.” In various implementations, the treatment planning system(s) 108 are managed by treatment technicians. As noted herein, the treatment plans may accommodate patient data in light of treatment preferences of users.

The treatment planning system(s) 108 may include engines that allow users of the treatment planning interface system(s) 104 to visualize, interact with, and/or fabricate appliances that implement a treatment plan. The treatment planning system(s) 108 may support UIs that display virtual representations of orthodontic appliances that move a patient's teeth from an initial position toward a final position to correct malocclusions of teeth. The treatment planning system(s) 108 can similarly include engines that configure the treatment planning interface system(s) 104 to display representations of restorative appliances and/or other medical appliances. The treatment planning system(s) 108 may support fabrication of appliances through, e.g., the appliance fabrication system(s) 110.

In some implementations, the treatment planning system(s) 108 provide customized GUIs that allow the user to discuss treatment plans with patients. The treatment planning system(s) 108 may render patient data, conditions to be treated, and/or treatment options for display on the treatment planning interface system(s) 104. The treatment planning system(s) 108 may render potential appliances that are prescribed to implement a treatment plan (e.g., series of orthodontic appliances that are configured to move a patient's dentition from a first position toward a target position in accordance with an orthodontic treatment plan; effects of specific appliances at various stages of a treatment plan, etc.).

The treatment planning system(s) 108 may include engines to support user interaction with treatment plans. The treatment planning system(s) 108 may use treatment preferences, including those generated in treatment domain-specific protocols by the CPM system(s) 106. In various implementations, the treatment planning system(s) 108 provide treatment templates to the treatment planning interface system(s) 104 that structure users' treatment preferences in a manner that is convenient to them. As noted herein, treatment templates may include structured data, UI elements (forms, text boxes, UI buttons, selectable UI elements, etc.), etc.

The treatment planning system(s) 108 may include one or more datastores configured to store treatment templates expressed according to treatment domain-specific protocols. The treatment planning system(s) 108 may further include one or more processing engines to process, e.g., parse, the treatment templates to form customized GUI elements on the treatment planning interface system(s) 104. As noted herein, the processing engines may convert the treatment templates into scripts or other runtime elements in order to support the customized GUI elements on the treatment planning interface system(s) 104. As noted herein, the treatment templates may have been created and/or validated by the CPM system(s) 106.

In some implementations, the treatment planning system(s) 108 provides the treatment planning interface system(s) 104 with customized GUI elements that are generated using treatment domain-specific protocols. The customized GUI elements may be based on treatment templates, which, for a user may be customized based on a template library of treatment templates for other users. The treatment templates may comprise public and/or private treatment In some implementations, the treatment planning system(s) 108 generates customized GUI elements for display by applications and/or processes on the treatment planning interface system(s) 104. Customized GUI elements may operate to display attributes of treatment plans that are relevant to a specific user.

The appliance fabrication system(s) 110 may include one or more computer systems configured to fabricate appliances. As discussed herein, examples of appliances to be fabricated include dental as well as non-dental appliances. Examples of dental appliances include aligners, other polymeric dental appliances, crowns, veneers, bridges, retainers, dental surgical guides, etc. Examples of non-dental appliances include orthotic devices, hearing aids, surgical guides, medical implants, etc.

The appliance fabrication system(s) 110 may comprise thermoforming systems configured to indirectly and/or directly form appliances. The appliance fabrication system(s) 110 may implement instructions to indirectly fabricate appliances. As an example, the appliance fabrication system(s) 110 may be configured to thermoform appliances over a positive or negative mold. Indirect fabrication of a dental appliance can involve one or more of the following steps: producing a positive or negative mold of the patient's dentition in a target arrangement (e.g., by additive manufacturing, milling, etc.), thermoforming one or more sheets of material over the mold in order to generate an appliance shell, forming one or more structures in the shell (e.g., by cutting, etching, etc.), and/or coupling one or more components to the shell (e.g., by extrusion, additive manufacturing, spraying, thermoforming, adhesives, bonding, fasteners, etc.). Optionally, one or more auxiliary appliance components as described herein (e.g., elastics, wires, springs, bars, arch expanders, palatal expanders, twin blocks, occlusal blocks, bite ramps, mandibular advancement splints, bite plates, pontics, hooks, brackets, headgear tubes, bumper tubes, palatal bars, frameworks, pin-and-tube apparatuses, buccal shields, buccinator bows, wire shields, lingual flanges and pads, lip pads or bumpers, protrusions, divots, etc.) are formed separately from and coupled to the appliance shell (e.g., via adhesives, bonding, fasteners, mounting features, etc.) after the shell has been fabricated.

The appliance fabrication system(s) 110 may comprise direct fabrication systems configured to directly fabricate appliances. As an example, the appliance fabrication system(s) 110 may include computer systems configured to use additive manufacturing techniques (also referred to herein as “3D printing”) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can include: (1) vat photopolymerization (e.g., stereolithography), in which an object is constructed layer by layer from a vat of liquid photopolymer resin; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances herein. In some embodiments, stereolithography involves selective polymerization of a photosensitive resin (e.g., a photopolymer) according to a desired cross-sectional shape using light (e.g., ultraviolet light). The object geometry can be built up in a layer-by-layer fashion by sequentially polymerizing a plurality of object cross-sections. As another example, the appliance fabrication system(s) 110 may be configured to directly fabricate appliances using selective laser sintering. In some embodiments, selective laser sintering involves using a laser beam to selectively melt and fuse a layer of powdered material according to a desired cross-sectional shape in order to build up the object geometry. As yet another example, the appliance fabrication system(s) 110 may be configured to directly fabricate appliances by fused deposition modeling. In some embodiments, fused deposition modeling involves melting and selectively depositing a thin filament of thermoplastic polymer in a layer-by-layer manner in order to form an object. In yet another example, the appliance fabrication system(s) 110 may be configured to implement material jetting to directly fabricate appliances. In some embodiments, material jetting involves jetting or extruding one or more materials onto a build surface in order to form successive layers of the object geometry.

In some embodiments, the appliance fabrication system(s) 110 may include a combination of direct and indirect fabrication systems. In some embodiments, the appliance fabrication system(s) 110 may be configured to build up object geometry in a layer-by-layer fashion, with successive layers being formed in discrete build steps. Alternatively or in combination, the appliance fabrication system(s) 110 may be configured to use a continuous build-up of an object's geometry, referred to herein as “continuous direct fabrication.” Various types of continuous direct fabrication systems can be used. As an example, in some embodiments, the appliance fabrication system(s) 110 may use “continuous liquid interphase printing,” in which an object is continuously built up from a reservoir of photopolymerizable resin by forming a gradient of partially cured resin between the building surface of the object and a polymerization-inhibited “dead zone.” In some embodiments, a semi-permeable membrane is used to control transport of a photopolymerization inhibitor (e.g., oxygen) into the dead zone in order to form the polymerization gradient. Examples of continuous liquid interphase printing systems are described in U.S. Patent Publication Nos. 2015/0097315, 2015/0097316, and 2015/0102532, (corresponding to U.S. patent Nos. corresponding to U.S. Pat. Nos. 9,205,601, 9,216,546, and 9,211,678) the disclosures of each of which are incorporated herein by reference in their entirety. As another example, the appliance fabrication system(s) 110 may be configured to achieve continuous build-up of an object geometry by continuous movement of the build platform (e.g., along the vertical or Z-direction) during the irradiation phase, such that the hardening depth of the irradiated photopolymer is controlled by the movement speed. Accordingly, continuous polymerization of material on the build surface can be achieved. Example systems are described in U.S. Pat. No. 7,892,474, the disclosure of which is incorporated herein by reference in its entirety.

In another example, the appliance fabrication system(s) 110 may be configured to extrude a composite material composed of a curable liquid material surrounding a solid strand. The composite material can be extruded along a continuous 3D path in order to form the object. Examples systems are described in U.S. Patent Publication No. 2014/0061974, corresponding to U.S. Pat. No. 9,511,543, the disclosures of which are incorporated herein by reference in its entirety.

In yet another example, the appliance fabrication system(s) 110 may implement a “heliolithography” approach in which a liquid photopolymer is cured with focused radiation while the build platform is continuously rotated and raised. Accordingly, the object geometry can be continuously built up along a spiral build path. Examples of such systems are described in U.S. Patent Publication No. 2014/0265034, corresponding to U.S. Pat. No. 9,321,215, the disclosures of which are incorporated herein by reference in its entirety.

The appliance fabrication system(s) 110 may include one or more elements of the aligner fabrication engine(s) 280 shown in FIG. 2.

The systems of the device planning environment 100 may operate to provide customized GUIs related to treatment planning. In some implementations, the treatment planning interface system(s) 104, the CPM system(s) 106 and the treatment planning system(s) 108 may operate to create treatment templates expressed according to treatment domain-specific protocols as follows. The CPM system(s) 106 may gather unstructured representations of treatment preferences from the treatment planning interface system(s) 104 through telephonic interviews, email exchanges, messages, conversations memorialized in notes, etc. A technician or an automated agent may use the tools on the CPM system(s) 106 to create treatment templates for a user in accordance with treatment domain-specific protocols. The CPM system(s) 106 may also validate the treatment templates to verify that the treatment templates accord with a given user and/or treatment outcome. The CPM system(s) 106 may provide the treatment templates to the treatment planning system(s) 108 for storage and/or use in execution.

Additionally, the treatment planning interface system(s) 104, the treatment planning system(s) 108, and/or the appliance fabrication system(s) 110 may operate to provide treatment plans and/or appliances for a given patient. As noted herein, the treatment planning interface system(s) 104 may gather patient data. With the patient data, a user whose treatment preferences were previously memorialized with a treatment template may gather one or more treatment plans using the engines in the treatment planning system(s) 108. The treatment planning system(s) 108 may gather treatment templates and parse these treatment templates using the treatment domain-specific protocols in order to efficiently and effectively generate customized GUI elements that express treatment preferences in the context of a treatment plan. The user may interact with the treatment plan using the treatment planning interface system(s) 104. In various implementations, the user and/or the treatment planning system(s) 108 provide instructions to fabricate appliances with the appliance fabrication system 110.

FIG. 2 is a diagram showing an example of a system 200; the system 200 may be incorporated into a portion of another system (e.g., a general treatment planning system) and may therefore also be referred to as a sub-system. In any of the method and apparatuses described herein, the system/sub-system may be invoked by a user control, such as a tab, button, etc., as part of treatment planning system, or may be separately invoked.

In FIG. 2, the system 200 may include a plurality of engines and datastores. A computer system can be implemented as an engine, as part of an engine or through multiple engines. As used herein, an engine may include one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices and need not be restricted to only one computing device. In some embodiments, the cloud-based engines can execute functionalities and/or modules that end users' access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

The system/sub-system 200 may include or be part of a computer-readable medium and may include a user interface (UF) engine 220. The user OF engine 220 may allow a user to interact with one or more software modules. As used herein, a user may refer to a doctor, dentist, or other clinician associated with determining, providing or generating a treatment plan. In some examples, the user OF engine 220 may display interactive dialog boxes to the user. In turn, the user may interact with the dialog boxes to indicate choices and/or decisions regarding a patient's treatment plan and/or a doctor's treatment preferences. In some variations, the dialog boxes may enable the user to modify part or portions of the patient's treatment plan and/or the doctor's treatment preferences. In some other variations, the user OF engine 220 may enable the user to modify the patient's treatment plan and/or the doctor/s treatment preferences within a data structure (e.g., a datastore). Thus, the user OF engine 220 may enable the user to read, modify, and/or write data within any feasible datastore. Alternatively, or in addition, the user OF engine 220 may enable the user to review, accept, reject, and/or apply any modifications to treatment preferences or treatment plans.

The system/subsystem 200 may also include a treatment plan generator engine 240. The treatment plan generator engine 240 may generate a treatment plan to provide dental or orthodontic treatment for a patient. In some examples, the treatment plan generator engine 240 may generate a patient's treatment plan based at least in part on patient data and a doctor's treatment preferences. Thus, the treatment plan generator engine 240 may accept or receive a data structure including treatment preferences and a digital model of the patient's teeth to generate a treatment plan. In some variations, the treatment plan generator engine 240 may store modified treatment parameters. The modified treatment parameters may be based on a doctor's treatment preferences as modified by the doctor (or any other feasible user) via the user OF engine 220. In some examples, the treatment plan generator engine 240 may store the treatment plan in an applicable datastore. Furthermore, the modified treatment parameters may be stored in a dental protocol language.

In some examples, the treatment plan generator engine 240 may determine or train one or more deep learning algorithms (often referred to as a deep learning network) for determining tooth apices of a patient. For example, the treatment plan generator engine 240 may provide supervised and/or unsupervised training of one or more deep learning networks based on patient data 230. The patient data 230 may include cone beam computed tomography (CBCT) scan data, panoramic radiograph data, and intraoral scan data. The training deep learning networks is described in more detail in conjunction with FIG. 3.

In some examples, the treatment plan generator engine 240 may execute the one or more deep learning networks described above. For example, the treatment plan generator engine 240 may process or apply patient data with any feasible deep learning network to determine tooth apices of a patient. In some variations, the patient data may include 2D panoramic radiograph data and/or 3D intraoral scan data. Execution of deep learning networks is described in more detail in conjunction with FIGS. 5 and 6.

The system/subsystem 200 may also include a display treatment plan engine 270. The display treatment plan engine 270 may enable the doctor to review and/or approve a patient's treatment plan, particularly a treatment plan as modified via the user OF engine 220. In some examples, the display treatment plan engine 270 may display before, during, and after representations or visualizations of a patient's teeth as treated by one or more aligners that are based on a treatment plan generated by the treatment plan generator engine 240.

The system/subsystem 200 may also include an aligner fabrication engine 280. The aligner fabrication engine 280 may process patient data 230 and a treatment plan to generate one or more (in some cases a series of) aligners, including clear dental aligners to treat a patient's teeth. The aligners may be generated by any feasible method. In some variations, the aligner fabrication engine 280 may generate images or renderings associated with one or more dental aligners. These images/renderings may be displayed to the user through the display treatment plan engine 270. In some examples, the aligners may be fabricated by the appliance fabrication system 110 of FIG. 1.

The system/subsystem 200 may include any number of datastores. For example, the system/subsystem 200 may include a data structure of treatment parameters 210. In some variations, the data structure may be expressed in a dental protocol language. The use of a dental protocol language (sometimes referred to as a domain-specific orthodontic treatment language) that is both human and machine readable and is tailored to orthodontic treatment provides a high level of flexibility and efficiency in orthodontic treatment planning and orthodontic device fabrications. For example, the dental protocol language enables automation of many different orthodontic treatment planning protocols, and facilitates the communication between users (e.g., doctors), technicians and R&D personnel. It adds more flexibility than simple parameter files because it includes semantics for conditional statement, and because it exposes more configuration options. The dental protocol language may be used for editing and for visualizing the treatment planning protocol (TPP) and may therefore be concise and easy to understand. The dental protocol language scripts may be automatically translated into executable code in an interpreted language.

The treatment parameters may describe a doctor's treatment preferences. In some examples, the treatment parameters may be predefined. For example, the doctor's treatment preferences may be based on a predefined personalized plan that may have been customized by and/or for the doctor to indicate one or more treatment preferences. In some examples, the doctor's treatment preferences may be predefined based on prior treatment plans approved by the doctor as used on other patients. In some other examples, the doctor's treatment preferences may be predefined based on those of a Key Opinion Leader (KOL). A Key Opinion Leader's treatment preferences may be those associated with a particular clinician. In some other examples, the doctor's treatment preferences may be predefined based on Regional Automated Defaults (RAD). RAD preferences may be associated with a geographic or other region with which the doctor wishes to follow. In still other examples, the doctor's treatment preferences may be predefined based on Dental Service Organization (DSO) templates. A DSO template may provide treatment preferences that are associated with and suggested by a doctor's DSO. In some examples, the predefined treatment parameters may be stored or maintained in a library of clinician treatment preferences. The treatment parameters may be stored, recorded, and/or indexed by the clinician.

The system/subsystem 200 may include a datastore of patient data 230. The patient data 230 may include any feasible patient data including scans, x-rays, dental imaging, patient physiological details (age, weight, gender), and the like. In some examples, patient data may include a digital model of the patient's teeth.

The system/subsystem 200 may include a datastore of modified treatment parameters 250. For example, the treatment plan generator engine 240 may store modified treatment parameters in the modified treatment parameter datastore 250. In some cases, the modified treatment parameters may be appended into the modified treatment parameter datastore 250. The treatment parameters may be modified through the user OF engine 220 interacting the data structure of treatment parameters 210.

The system/subsystem 200 may include a treatment plan datastore 260. The treatment plan datastore 260 may include a treatment plan for a patient based on treatment parameters in the modified treatment parameter datastore 250.

Knowing specific points of a patient's teeth may assist a clinician in determining a patient's treatment plan. Geometries of the patient's teeth may be visible (e.g., above the gum line) and occluded (e.g., below the gum line). In particular, the tooth root apices are typically well below the gum line and may greatly affect tooth mobility and responsiveness to treatment, such as orthodontic treatment.

An accurate 3D picture of a patient's teeth, including both crown and root, can be obtained using CBCT, but such studies are not always available due to the high cost of CBCT scanners and a desire to minimize patient x-ray exposure. However, 2D panoramic radiographs are often captured by orthodontists. A 2D panoramic radiograph can provide a flat image of a patient's teeth and adjacent bone structure. Also, 3D intraoral scanning is frequently used to provide 3D information of the visible portion of the patient's teeth. Since 3D intraoral scanning uses visible light, exposure to harmful x-rays is minimized. However, any tooth root information may be hidden by the gums.

In some examples, a convolutional neural network (CNN) may be trained to implement a deep learning network that may be used to determine tooth root apices when provided 2D radiograph data and/or 3D intraoral scan data. In this manner, tooth root information may be determined without access to a patient's CBCT scans. Thus, a more accurate treatment plan may be provided to a patient without access to CBCT scan data.

FIG. 3 is a flowchart showing an example method 300 for training a deep learning network for determining coordinates of tooth root apices. Some examples may perform the operations described herein with additional operations, fewer operations, operations in a different order, operations in parallel, and some operations differently. The method 300 is described below with respect to the system 200 of FIG. 2, however, the method 300 may be performed by any other suitable system or device.

The method 300 begins in block 302 as the system 200 obtains CBCT scan data. The CBCT scan data may be used to train the deep learning network. The CBCT scan data may include comprehensive 3D tooth and bone information for a plurality of patients. In some examples, the 3D tooth information may include coronal tooth data and root tooth information. Because CBCT scan data is collected with x-rays, accurate tooth data may be collected, including tooth root data typically occluded by the patient's gums. In some examples, the CBCT scan data may include 3D representations using 3D voxel volumes of various teeth.

In some examples, the CBCT scan data may be segmented into semantically segmented dental structures (e.g., teeth and bones). The segmented dental structures may include individual representations of a tooth and jaw and include a representation as a 3D mesh. Segmentation may be performed manually (e.g., by a user) or automatically (e.g., by a processor executing a segmentation algorithm). In some examples, the segmented dental structures may include labels associated a tooth number with a particular tooth. For example, a tooth number may identify a particular tooth within a patient's dental arch. Coordinates of one or more tooth root apices information for each tooth may be determined. For example, for the CBCT scan data, the system 200 may calculate or determine root apex information for each tooth within the segmented CBCT scan.

Next, in block 304 the system 200 obtains 2D panoramic radiograph data corresponding to the CBCT scan data described in block 302. The 2D panoramic radiograph data may be used to train the deep learning network. The 2D panoramic radiograph data may include a large section of the facial skull in conditions similar to those present in the CBCT scan data. In some variations, there may be a one-to-one correspondence between CBCT scan data and 2D panoramic radiograph data. In some cases, the 2D panoramic radiograph data may be compared to the corresponding CBCT scan data to determine and ensure that both data sets share a common coordinate system and that the data sets match each other within a tolerance amount. For example, tooth apex coordinate information in the 2D panoramic radiograph data and the CBCT scan data may be inspected to ensure that they are each within a predetermined distance of each other.

Next, in block 306 the system 200 obtains 3D intraoral scan data corresponding to the CBCT scan data described in block 302. The 3D intraoral scan data may be used to train the deep learning network. The 3D intraoral scan data may include 3D tooth data, particularly tooth data associated with the tooth visible above the gumline. There may be a one-to-one correspondence between CBCT scan data and 3D intraoral scan data. In some examples, the 3D intraoral scan data may include tooth, gum, palate, and other physical structures as a 3D mesh. In some cases, the 3D intraoral scan data may be compared to the corresponding CBCT scan data to determine and ensure that both data sets share a common coordinate system and that the data sets match each other within a tolerance amount. For example, tooth apex coordinate information in the 3D intraoral scan data and the CBCT scan data may be inspected to ensure that they are each within a predetermined distance of each other.

Next, in block 308 the system 200 determines a 2D CNN. Determination of the 2D CNN may include training of the 2D CNN using the CBCT scan data (from block 302), 2D the panoramic radiograph data (from block 304), and the 3D intraoral scan data (from block 306). In some examples, the training may be a supervised training that determines a relationship between the location of any particular tooth apex from the CBCT data with respect to the 2D panoramic radiograph data and the 3D intraoral scan data. In particular, training the 2D CNN may include determining and minimizing a loss function (e.g., a cost function) between an actual tooth apex (e.g., ground truth data determined from the CBCT data) and a predicted tooth apex based on the 2D panoramic radiograph scan data. Training data of the 2D CNN may include tooth number information from the CBCT scan data. The resulting 2D CNN may be used to estimate locations of a patient's tooth root apex based on a patient's 2D panoramic radiograph data.

Next, in block 310, the system 200 determines a 3D CNN. Determination of the 3D CNN may include training of the 3D CNN using CBCT data, the 2D panoramic radiograph data, and the 3D intraoral scan data. In some examples, the training may be a supervised training that determines a relationship between the location of any particular tooth apex from the CBCT data with respect to the 2D panoramic radiograph scan data and the 3D intraoral scan data. In particular, determining the 3D CNN may include determining and minimizing a loss function between an actual tooth apex (e.g., ground truth data determined from the CBCT data) and a predicted tooth apex based on the 3D intraoral scan data. Training data of the 3D CNN may include tooth number information from the CBCT scan data. The resulting 3D CNN may be used to estimate locations of a patient's tooth root apex based on a patient's 3D intraoral scan data.

Next, in block 312, the system 200 determines geometric tooth parameters. This block may be optional, as denoted with dashed lines in FIG. 3. For example, for each CBCT scan data, 2D panoramic radiograph data, and 3D intraoral scan data, the system 200 can determine a set of geometric tooth parameters that describe the patient's tooth and dental arch. Example geometric tooth parameters may include tooth crown radius, crown tip point, arch diameter, and the like.

Next, in block 314, the system determines a geometric tooth CNN. The geometric tooth CNN may be trained using CBCT scan data and the geometric tooth parameters. In particular, the geometric tooth parameters may be limited to visible (above the gum line) parameters. Training data of the geometric tooth CNN may include tooth number information from the CBCT scan data. The resulting geometric CNN may be used to estimate locations of a patient's tooth root apex based on a geometric tooth data.

FIG. 4 shows an example of an intraoral scan matched with a segmented CBCT scan 400. In addition, FIG. 4 shows a computed tooth root apex 410 for one tooth. Although only one tooth root apex 410 is shown, in other examples, the 2D CNN and/or the 3D CNN may compute the tooth root apex for any feasible tooth. The tooth root apex 410 may be provided by the 2D CNN and/or the 3D CNN.

FIG. 5 is a block diagram showing data and process flow of a deep learning network 500 configured to determine tooth root apex coordinates 590 of a patient's teeth. The deep learning network 500 may include a 2D CNN 510 (sometimes referred to as a Regression 2D CNN) and a 3D CNN 520 (sometimes referred to as a Regression 3D CNN). The 2D CNN 510 may be an example of the 2D CNN described with respect to block 308 of FIG. 3. Similarly, the 3D CNN 520 may be an example of the 3D CNN described with respect to block 310 of FIG. 3.

In some examples, the process may begin with processing a patient's 2D panoramic radiograph data 504 (sometimes referred to as 2D panoramic X-ray data). The 2D panoramic radiograph data 504 may be processed by the 2D CNN 510. In this manner, the deep learning 2D CNN 510 may provide a first set of the patient's tooth root apex coordinates 511.

In some other examples, the process may begin with processing a patient's 3D intraoral scan data 502. The 3D intraoral scan data 502 may be processed by the 3D CNN 520. In this manner, the deep learning 3D CNN 520 may provide a second set of the patient's tooth root apex coordinates 521.

Notably, the process flow may use either the 2D panoramic radiograph data 504 or the 3D intraoral scan data 502. In other words, the process flow may not require both the 2D panoramic radiograph data 504 and the 3D intraoral scan data 502 to determine the patient's tooth root apex coordinates. However, both the 2D panoramic radiograph data 504 and the 3D intraoral scan data 502 may be used provided they are both available.

The first and second set of tooth root apex coordinates 511 and 521 may be combined at combiner 530. The combiner 530 may combine the first and second set of tooth root apex coordinates 511 and 521 using any feasible method including, but not limited to, averaging, interpolating, weighted averaging, and the like.

A tooth number 506 may be supplied by the user. The tooth number may indicate a particular tooth for which tooth root apex coordinates are desired. The tooth number may be processed by an embedding block 540. The embedding block 540 combines one-hot vector encoding and full-connected layer, so output of the embedding block 540 is a vector of float values.

From the embedding block 540, the tooth number information is provided to an attention block 550. In neural networks (such as the 2D CNN and the 3D CNN), attention is a technique that mimics cognitive attention. The effect improves some parts of the input data and reduces other parts—the idea is that the network should pay more attention to this small but important part of the data. Learning which piece of data is more important than others may be context dependent and is trained using gradient descent. Output of the embedding block passes to input of attention block 550 where vector of size equal to size of Intraoral and panoramic features addition block output is computed. Values in the resulting vector are in range [0 . . . 1], where larger value corresponds to more importance of the feature on the output of intraoral and panoramic features addition block.

Output from the attention block 550 is provided to an attenuation block 560. The attenuation block 560 can use the output of the attention block 550 to weight data from the combiner block 530 (e.g., tooth apex information from the 2D CNN 510 and/or the 3D CNN 520). For example, output of the combiner block 530 may be multiplied by attention block 550 providing a vector of features including intraoral and panoramic radiograph features attenuated in correspondence with importance of the dedicated feature (e.g., selected tooth number 506).

Output from the attenuation block 560 is provided to an addition block 570. In the addition block 570, outputs of block of the attenuation block 560 (e.g., attenuated intraoral and panoramic radiograph features) and the embedding block 540 are added to generate a final set of features including all available information about the tooth number, intraoral scan containing this tooth number and panoramic radiograph containing this tooth number.

Output from the addition block 570 is provided to a neural network 580. In some examples, the addition block 570 provides a final set of feature feeds to a neural network 580. The neural network generates tooth root apex coordinates 590 with respect to the tooth number 506.

FIG. 6. is a flowchart showing an example method 600 for determining tooth root apex coordinates via a deep learning network. The method 600 is described below with respect to the system 200 of FIG. 2, however, the method 600 may be performed by any other suitable system or device.

The method 600 begins in block 602 as the system 200 obtains (or retrieves from a memory) patient data 230. In some examples, the patient data 230 may include 2D panoramic radiograph data and/or 3D intraoral scan data. In some variations, the patient data 230 may include either 2D panoramic radiograph data or 3D intraoral scan data, but not both.

Next, in block 604 the system 200 applies or provides the patient data 230 to one or more deep learning networks to obtain tooth root apex coordinates. For example, the system 200 may provide 2D panoramic radiograph data 504 of FIG. 5 to the 2D CNN 510 to infer root tooth apex coordinates from the 2D data. In another example, the system 200 may provide 3D intraoral scan data 502 to the 3D CNN 520 to infer root tooth apex coordinates from the 3D data. In some variations, the tooth root apex coordinate information from the 2D CNN 510 may be combined with tooth root apex coordinate information from the 3D CNN 520.

Next, in block 606 the system 200 may obtain a tooth number 506. In some examples, the tooth number 506 may indicate a particular tooth for which a user or clinician wishes to determine associated tooth root apex coordinates.

Next, in block 608 the system 200 may determine the tooth root apex coordinates associated with the tooth number obtained in block 606. In some embodiments, the tooth number may be used to weight tooth apex coordinates from the 2D CNN 510 and/or the 3D CNN 520. In some examples, the system 200 may determine the tooth root apex coordinates in accordance with the data and process flow of the deep learning network 500 of FIG. 5.

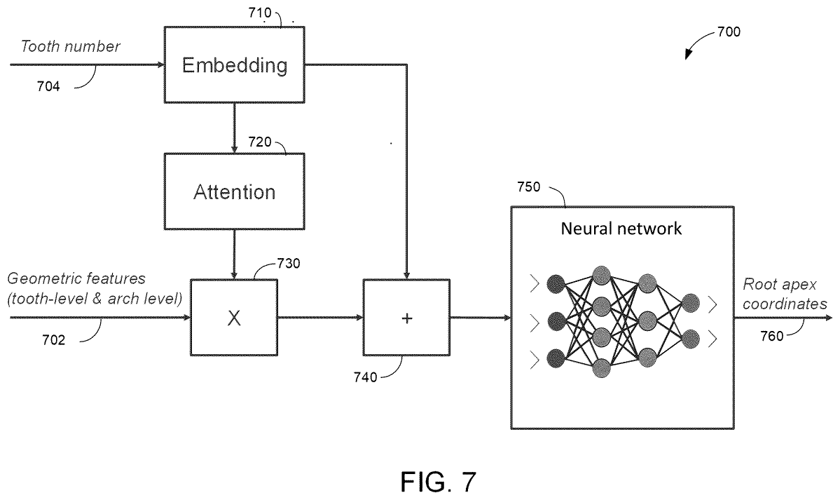

In some examples, tooth root apex coordinates may be determined based on a patient's dental (tooth) geometric features. This is described in more detail with respect to FIG. 7.

FIG. 7 is a block diagram showing data and process flow of a deep learning network 700 configured to determine tooth root apex coordinates 760 of a patient's teeth. The deep learning network may include a geometric CNN as described with respect to FIG. 3. In some examples, the process may begin with receiving a tooth number 704. The tooth number 704 may specify a particular tooth for which tooth root apex coordinates are desired.

The tooth number 704 may be processed by an embedding block 710 and an attention block 720. The embedding block 710 may be an example of the embedding block 540 of FIG. 5. Similarly, the attention block 720 may be an example of the attention block 550.

The deep learning network 700 may receive geometric features 702 of a patient's teeth. The geometric features 702 may include tooth-level geometric descriptors and, in some variations, may include dental arch information. The geometric features 702 may include tooth crown radius, crown tip point, arch diameter and the like.

An attenuation block 730 may operate on the geometric features 702. For example, certain geometric features 702 may be weighted to be enhanced or attenuated. Output from the attenuation block 730 is provided to an addition block 740. Output of the embedding block 710 and output from the attenuation block 730 may be used by the addition block 740 to provide a final set of feature feeds to a neural network 750. Output of the neural network 750 may include the tooth root apex coordinates 760.

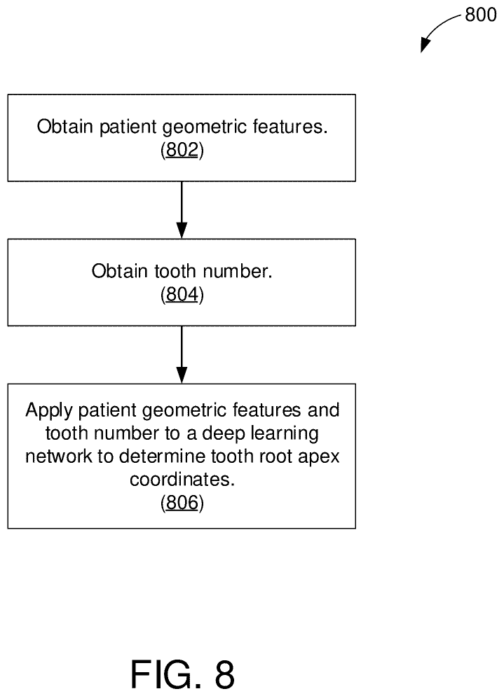

FIG. 8. is a flowchart showing an example method 800 for determining tooth root apex coordinates via a deep learning network. The method 800 is described below with respect to the system 200 of FIG. 2, however, the method 800 may be performed by any other suitable system or device. The method begins in block 802 as the system 200 obtains patient geometric features. The geometric features may include any feasible tooth-level and/or dental arch level geometric features, details, and/or characteristics of a patient's teeth. In some variations, the geometric features may be stored in the patient data 230.

Next, in block 804 the system 200 may obtain a tooth number. In some examples, the tooth number may indicate a particular tooth for which a user or clinician wishes to determine an associated tooth root apex coordinates.

Next, in block 806 the system 200 may apply patient geometric features and a tooth number to a deep learning network to determine tooth root apex coordinates. For example, the system may execute a geometric tooth CNN to determine one or more tooth root apices.

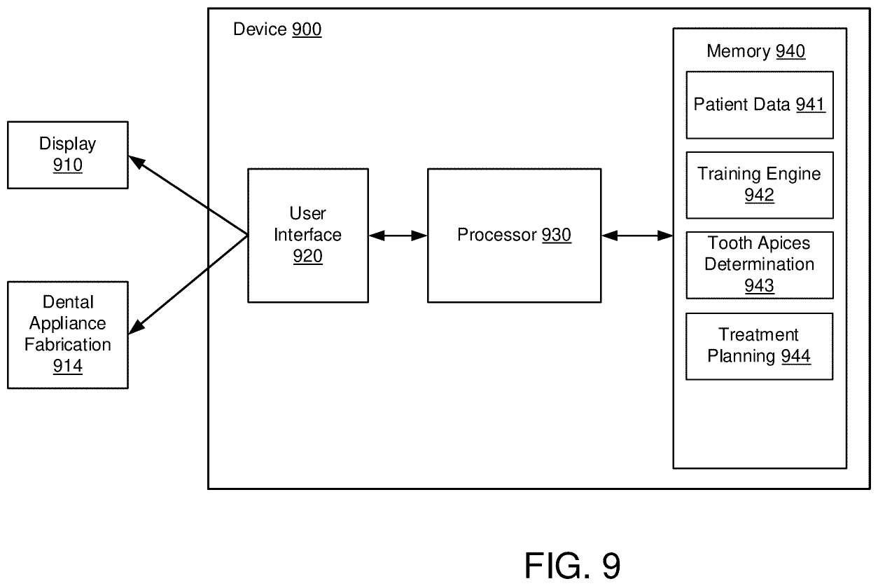

FIG. 9 shows a block diagram of device 900 that may be one example of a device that may be configured to perform any of the operations described herein. The device 900 may include a user interface 920, a processor 930, and a memory 940. The device 900 may be local (near) the user (clinician) that wants to determine dental appliance (e.g., dental aligner) data. In some variations, the device 900 may be remote (separate) from the user. For example, the device 900 may be implemented as a server or may be distributed on two or more servers or may be cloud (internet) based.

The user interface 920, which is coupled to the processor 930, may be used to interface with any device that receives or transmit data to and/or from the device 900. For example, the user interface 920 may be coupled to a display 910. The display 910 may show the user predicted, desired, and original tooth positions. The display 910 may be included on a mobile device such as a smart phone, a tablet computer, or laptop. The display 910 also may be included on devices that are not conventionally mobile such as a desktop computer or wall mounted display screen.

The user interface 920 may be coupled to a dental appliance fabrication unit 914. The dental appliance fabrication unit 914 may receive dental appliance data generated by the device 900 and, in turn, generate dental appliances. In some cases, the dental appliance data from the device 900 may be used to generate dental aligners, including clear dental aligners.

The user interface 920 may receive patient data 941 which, in turn, may be stored in the memory 940. The patient data may include 2D panoramic radiograph data, 3D intraoral scan data, and CBCT scan data.

The processor 930, which is also coupled to the memory 940, may be any one or more suitable processors capable of executing scripts or instructions of one or more software programs stored in the device 900 (such as within memory 940).

The memory 940 may also include a non-transitory computer-readable storage medium (e.g., one or more nonvolatile memory elements, such as EPROM, EEPROM, Flash memory, a hard drive, etc.) that may store the following software modules:
a training engine 942 to train one or more convolutional neural networks;
a tooth apices determination module 943 to determine a patient's tooth apices; and
a treatment planning module 944 to determine patent treatment plans and generate dental appliance data.
   Each software module, module, or engine includes program instructions that, when executed by the processor 930, may cause the device 900 to perform the corresponding function(s). Thus, the non-transitory computer-readable storage medium of memory 940 may include instructions for performing all or a portion of the operations described herein.

The processor 930 may execute the training engine 942 to train one or more deep learning convolutional neural networks (CNNs). For example, execution of the training engine 942 may use the patient data 941 to train a 2D CNN, a 3D CNN, and/or a geometric tooth CNN. In some examples, execution of the training engine 942 may use the CBCT scan data as reference ground truth information. In some variations, execution of the training engine 942 may cause the processor 930 to determine and minimize a cost function with respect to the CBCT scan data and other patient data.

The processor 930 may execute the tooth apices determination module 943 to determine one or more tooth root apices of a patient. In some variations, execution of the tooth apices determination module 943 may cause the processor 930 to execute a CNN such as a 2D CNN, a 3D CNN, a geometric tooth CNN, or the like. For example, 2D panoramic radiograph data may be provided to a 2D CNN to determine one or more tooth root apices. In another example, 3D intraoral scan data may be provided to a 3D CNN to determine one or more tooth root apices. In still another example, the processor 930 may provide tooth geometries to a geometric tooth CNN to determine one or more tooth root apices.

The processor 930 may execute the treatment planning module 944 to determine treatment plans for a patient and determine dental appliance data. For example, execution of the treatment planning module 944 may cause the processor 930 to determine a dental treatment plan for a patient based on tooth root apieces determined by the tooth apices determination module 943. The processor 930 may execute the treatment planning module 944 to generate the dental appliance data that, in turn, may be used to generate dental appliances. The dental appliance data may correspond to a determined treatment plan.

In some variations, the dental appliance data may be used to generate an image that may be displayed to the user. For example, an image of the predicted final tooth position may be displayed based on the dental appliance data. In some other variations, the predicted final tooth position may be superimposed with a patient's beginning or initial tooth position on the display 910.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of generating a treatment plan for forming one or more dental appliances by determining coordinates of a tooth root apex, the method comprising:
obtaining patient data, wherein the patient data includes at least one of two-dimensional (2D) panoramic radiograph data of a patient and three-dimensional (3D) intraoral scan data of the patient;
obtaining a tooth number;
providing the patient data and the tooth number to a trained deep learning network; and
determining, via a processor executing the trained deep learning network, the coordinates of a tooth root apex based on the patient data and corresponding to the tooth number;
generating or modifying the treatment plan using the coordinates of the tooth root apex; and
forming one or more dental appliances according to the treatment plan.

2. The method of claim 1, wherein the deep learning network includes a 2D convolutional neural network configured to determine the coordinates of the tooth root apex from the 2D panoramic radiograph data of the patient.

3. The method of claim 2, wherein the 2D convolutional neural network is trained based at least in part on cone beam computed tomography (CBCT) tooth data.

4. The method of claim 1, wherein the deep learning network includes a 3D convolutional neural network configured to determine the coordinates of the tooth root apex from the 3D intraoral scan data of the patient.

5. The method of claim 4, wherein the 3D convolutional neural network is trained based at least in part on CBCT tooth data.

6. The method of claim 1, wherein the tooth number selectively weights an output of the deep learning network.

7. The method of claim 1, wherein the deep learning network determines the coordinates of the tooth root apex based on the 2D panoramic radiograph data of the patient and the 3D intraoral scan data of the patient.

8. The method of claim 1, wherein the deep learning network is trained based at least in part on cone beam computed tomography (CBCT) tooth data and 2D panoramic radiograph data corresponding to the CBCT tooth data.

9. The method of claim 1, wherein the deep learning network is trained based at least in part on CBCT tooth data and 3D intraoral scan data corresponding to the CBCT tooth data.

10. A system, the system comprising:
a treatment plan generator engine configured to:
obtain, from a memory, patient data, wherein the patient data includes at least one of two-dimensional (2D) panoramic radiograph data of a patient and three-dimensional (3D) intraoral scan data of the patient;
obtain, from the memory, a tooth number; and
provide the patient data and the tooth number to a trained deep learning network; and
a processor configured to determine, via the trained deep learning network, coordinates of a tooth root apex based on the patient data and corresponding to the tooth number,
wherein the treatment plan generator is configured to use the coordinates of the tooth root apex to generate or modify a treatment plan.

11. The system of claim 10, further comprising an appliance fabrication subsystem configured to fabricate one or more appliances from the treatment plan.

12. A non-transitory computer-readable storage medium comprising instructions that, when executed by one or more processors of a device, cause the device to perform operations comprising:
obtaining patient data, wherein the patient data includes at least one of two-dimensional (2D) panoramic radiograph data of a patient and three-dimensional (3D) intraoral scan data of the patient;
obtaining a tooth number;
providing the patient data and the tooth number to a trained deep learning network; and
determining, via a processor executing the trained deep learning network, the coordinates of a tooth root apex based on the patient data and corresponding to the tooth number;
generating or modifying the treatment plan using the coordinates of the tooth root apex; and
forming one or more dental appliances according to the treatment plan.

13. The non-transitory computer-readable storage medium of claim 12, wherein the deep learning network includes a 2D convolutional neural network configured to determine the coordinates of the tooth root apex from the 2D panoramic radiograph data of the patient.

14. The non-transitory computer-readable storage medium of claim 13, wherein the 2D convolutional neural network is trained based at least in part on cone beam computed tomography (CBCT) tooth data.

15. The non-transitory computer-readable storage medium of claim 12, wherein the deep learning network includes a 3D convolutional neural network configured to determine the coordinates of the tooth root apex from the 3D intraoral scan data of the patient.

16. The non-transitory computer-readable storage medium of claim 15, wherein the 3D convolutional neural network is trained based at least in part on CBCT tooth data.

17. The non-transitory computer-readable storage medium of claim 12, wherein the tooth number selectively weights an output of the deep learning network.

18. The non-transitory computer-readable storage medium of claim 12, wherein the deep learning network determines the coordinates of the tooth root apex based on the 2D panoramic radiograph data of the patient and the 3D intraoral scan data of the patient.

19. The non-transitory computer-readable storage medium of claim 12, wherein the deep learning network is trained based at least in part on cone beam computed tomography (CBCT) tooth data and 2D panoramic radiograph data corresponding to the CBCT tooth data.

20. The non-transitory computer-readable storage medium of claim 12, wherein the deep learning network is trained based at least in part on CBCT tooth data and 3D intraoral scan data corresponding to the CBCT tooth data.
